(19) United States
(12) Patent Application Publication
(10) Pub. No.: US 2026/0256834 A1
(43) Pub. Date: Sep. 3, 2026

(54) Materials and Methods for Reducing the Occurrence and/or Severity of Autism Spectrum Disorder

(71) Applicant: FLAASK, LLC, University Heights, OH (US)

(72) Inventors: KEN ALIBEK, Dunn Loring, VA (US); ANDREW R. LEFKOWITZ, University Heights, OH (US)

(21) Appl. No.: 19/164,950

(22) PCT Filed: Mar. 13, 2024
(86) PCT No.: PCT/US2024/019717

§ 371 Date: Sep. 12, 2025

Related U.S. Application Data
(60) Provisional application No. 63/489,870, filed on Mar. 13, 2023.

(51) Int. Cl. A61K 33/30; A61K 31/05; A61K 31/12; A61K 31/198; A61K 31/4525; A61K 31/522; A61K 33/04; A61K 33/06; A61K 35/744; A61K 36/068; A61K 36/16; A61K 36/88

(52) U.S. Cl. CPC A61K 33/30; A61K 31/05; A61K 31/12; A61K 31/198; A61K 31/4525; A61K 31/522; A61K 33/04; A61K 33/06; A61K 35/744; A61K 36/068; A61K 36/16; A61K 36/88

(57) ABSTRACT

The present invention provides compositions and methods for improving the quality of life for a subject, particularly a child, diagnosed with autism spectrum disorder (ASD). In preferred embodiments, a supplement composition comprising natural or naturally-derived ingredients is delivered to a subject in the form of a chocolate bar or other candy alongside administration of an antiviral medication. Advantageously, the composition and methods can improve the immune health of the subject, along with other signs and symptoms associated with ASD, infections and other immunocompromising conditions.

### CROSS-REFERENCE TO RELATED APPLICATION

This application claims priority to U.S. Provisional Patent Application No. 63/489,870, filed Mar. 13, 2023, which is incorporated herein by reference in its entirety.

### BACKGROUND OF INVENTION

Autism spectrum disorder (ASD) refers to a group of neurodevelopment disorders, including autistic disorder (autism), Asperger syndrome, childhood disintegrative disorder, and pervasive developmental disorders, which are characterized by repetitive, distinctive patterns of behavior and difficulties with social communication and interaction. Subjects with ASD may have a combination of many symptoms and syndromes, which are typically present from early childhood and affect daily functioning.

Included within ASD is a wide range, or spectrum, of symptoms, skills, and levels of functional disability. Some children and adults with ASD are fully capable of performing all activities of daily life, while others require substantial support to perform basic life functions and activities. Additionally, individuals with ASD may experience pathologic changes such as brain inflammation, gastrointestinal problems, immune system imbalance, lipid metabolism imbalance, and possible increased risk of cancer due to links between autism and mutations in cancer-associated genes and pathways.

The prevalence of ASD in children in the United States has been increasing rapidly over the past 50 years. Considerably more males are affected with ASD than females. For some individuals, the core symptoms of ASD (i.e., impairments in communication and social interaction, and restricted/repetitive behaviors and interests) may improve with intervention and maturation; however, core deficits typically translate into varying developmental presentations that endure throughout the lifespan.

The exact cause of ASD development is not entirely clear, and the underlying root of ASD symptoms, such as damaged brain structures and impaired nerve connections, cannot be readily corrected by existing medications. However, drugs useful in treating other diseases with similar symptoms can be useful in managing the manifestations of ASD, though none have been approved by the FDA for treating ASD specifically.

There is no cure for ASD, but current therapies and behavioral interventions exist, which are designed to improve certain symptoms of the disorder; however, most existing treatments focus on treating the brain and cognitive functions specifically, rather than the other potential underlying causes of ASD and its symptoms, for example, compromised immune health.

The ideal treatment plan for a subject diagnosed with ASD coordinates therapies and interventions that meet the specific needs of the individual, based on where he or she falls on the spectrum. Most health care professionals agree that the earlier the intervention, the better.

Thus, there is a continuing need for new, integrated compositions and methods for reducing the occurrence and/or severity of ASD in children, as well as treating a broad range of ASD symptoms to improve the overall quality of life and performance for patients diagnosed with autism and its spectra.

### BRIEF SUMMARY

The present invention provides compositions and methods for identifying individuals at risk for developing autism spectrum disorder (ASD), for reducing the likelihood of developing ASD, and for treating people with ASD. Advantageously, embodiments of the present invention provide compositions and methods for improving the quality of life for subjects, particularly children, who have developed, or who are at risk for developing, ASD.

The materials and methods of the subject invention are based, in part, on the identification of an etiological progression associated with the development of ASD, including the identification of a sequence of alterations and damage to different physiological systems and organs in a subject. These alterations and damage lead to the development, progression, and perpetuation of ASD, and associated symptoms and conditions.

In one specific embodiment, the present invention provides materials and methods for intervening in an infectious etiology wherein, pursuant to this etiology, first, a fetus is exposed to a pathogen, such as a virus or bacteria, or the fetus is exposed to maternal factors associated with the mother having been exposed to a pathogen. This exposure of the fetus to a pathogen and/or to deleterious immune system factors, induces inflammation and/or other deleterious immune responses in the fetus, which then elicit oxidative stress and/or pro-inflammatory factors that can cause DNA alterations, folate deficiency and/or damage to the gut-brain axis in the fetus or newborn child. These alterations cause abnormal brain development in the fetus and/or child and ultimately result in ASD. Further, these physiological changes compromise the immune system of the subject such that the subject cannot effectively fight the persistent infection, and the cycle repeats itself causing progression and perpetuation of autistic symptoms and ASD-related disorders.

In certain embodiments, the present invention provides a multi-step method for treating a subject with ASD with the goal of shifting the subject “up” on the autism spectrum to a less severe manifestation of the disorder. The method can comprise (1) testing a subject for, and/or diagnosing the subject with, ASD and/or a risk marker of ASD; (2) analyzing the test results and determining a course of treatment based thereon; and (3) administering the course of treatment to the subject.

In some embodiments, the method can further comprise periodically assessing the effectiveness of the treatment; and, based on the periodic assessment, adjusting the treatment as appropriate.

In one embodiment, the method can be used to treat and/or reduce the severity of symptoms of ASD, including behavioral, mental, emotional and/or physiological symptoms. The subject can be any human diagnosed with ASD. In one embodiment, the human subject is a child or adolescent, for example, 16 years of age or younger.

In certain embodiments, the present invention provides a multi-step method for preventing and/or reducing the severity of in utero development of ASD in a fetal or neonatal subject. The method can comprise (1) testing a woman who is pregnant, or who plans to become pregnant, for ASD risk markers as identified herein; (2) analyzing the test results and determining a course of treatment for the woman based thereon; and (3) administering the course of treatment to the woman.

In some embodiments, the method can further comprise periodically assessing the effectiveness of the treatment; and, based on the periodic assessment, adjusting the treatment for the woman as needed throughout the pregnancy and afterwards (e.g., throughout the time period that the child is nursing).

In certain embodiments, the woman has a personal or family history of ASD or other neurodevelopmental conditions. In certain embodiments, the woman has previously given birth to one or more children diagnosed with ASD.

In one embodiment, the method can be used to reduce the likelihood that a woman will give birth to a child with ASD, and/or reduce the severity of symptoms that occur in a child born with ASD, including behavioral, mental, emotional and/or physiological symptoms.

According to the subject invention, testing in part (1) of the methods can comprise, for example, conducting a comprehensive blood test to determine the presence and extent of risk markers, either in the subject, or in a woman who is pregnant or plans to become pregnant.

Testing can include CARS™ (Childhood Autism Rating Scale) testing, gastrointestinal system testing (e.g., stool sampling) for gut microbiome analysis, reviewing personal and family medical history, and in the case of pathogenic infections that cause, e.g., external lesions, sampling, culturing and polynucleotide sequencing.

In certain embodiments, the risk markers according to the subject invention can include signs of poor immune health, for example, immune markers such as T-cells and NK cells.

In certain embodiments, the risk markers are antibodies indicating a previous or current infection or exposure to an infection agent selected from one or more of cytomegalovirus (CMV), Epstein-Barr virus (EBV), rubella virus, measles virus, herpes simplex virus (HSV) type 1 or 2, herpes zoster, varicella-zoster virus (VZV), HHV6, HHV7, other herpes family viruses, Mycoplasma, Chlamydia, Helicobacter, Toxoplasma, or any other chronic, congenital, persistent, latent, dormant, acute and/or subacute infection. In preferred embodiments, the infection is due to a TORCH infection (Toxoplasmosis, Other (Syphilis, Parvovirus B19, HIV, HBV, and Varicella zoster), Rubella, CMV and/or HSV).

In one embodiment, the expectant mother, neonate and/or child has an abnormal microbiota, e.g., Clostridial overgrowth, Staphylococcus overgrowth, Candida overgrowth, Aspergillus overgrowth, SIBO, SIFO, or any biofilm-related disease.

In one embodiment, the expectant mother and/or child has an autoimmune disorder, such as, for example, diabetes, psoriasis, lupus, Cron's, vitiligo, Hashimoto's hypothyroidism, Addison's disease, and others.

After determining the extent of risk factors present in the expectant mother, fetus, neonate and/or child, the methods of the present invention can further comprise treating the expectant mother, fetus, neonate and/or child to reduce the occurrence and/or severity of ASD. Preferably, treatment comprises a combination of prescription medications and/or over-the-counter medications with a unique blend of supplement formulations that address various risk factors, including, e.g., infection, poor digestive and/or mucosal health, poor immune health, metabolic conditions, endocrine disruption and inflammation.

In some embodiments, treatment further comprises prescribing a specified diet regimen to an expectant mother or a child, wherein the diet has therapeutic effects toward the prevention and/or improvement of ASD symptoms. In one embodiment, the diet is a low-sugar or low-carbohydrate diet, such as the “Keto Diet” or “Atkins® Diet.”

In certain embodiments, treatment is intermittently adjusted based on behavioral and medical improvements until a maintenance “supplements only” treatment is achieved. In some embodiments, assessment involves CARS

In certain embodiments, the present invention provides supplement formulations with specified functions, which are useful according to the methods described herein. The specified functions include (1) suppression of low-grade inflammation and reactive molecules; (2) recovery of normal mucosa and mucosal immunity; (3) establishment of healthy microbiota and immunity restoration; (4) treatment and modulation of metabolic and endocrine abnormalities; (5) restoration of normal neural functions and enhancement of neurogenesis; and/or (6) recovery and “fine-tuning” of emotional and cognitive spheres. The supplement formulations are provided to a subject in a pre-determined cascading order to provide cumulative therapeutic benefits.

Advantageously, the present invention can improve the quality of life for subjects who are diagnosed with, for example, ASD or another neurodevelopmental condition. Improved quality of life can include, for example, improvement of mental, emotional and/or physiological symptoms of ASD, improvement in behavioral performance, and improvement in signs and symptoms of infections.

### DETAILED DISCLOSURE

The present invention provides compositions and methods for identifying individuals at risk for developing autism spectrum disorder (ASD), for reducing the likelihood of developing ASD, and for treating people with ASD. Advantageously, embodiments of the present invention provide compositions and methods for improving the quality of life for subjects, particularly children, who have developed, or who are at risk for developing, ASD.

The materials and methods of the subject invention are based, in part, on the identification of the etiological progression associated with the development of ASD, including the identification of a sequence of alterations and damage to different physiological systems and organs in a subject. These alterations and damage lead to the development, progression, and perpetuation of ASD, and associated symptoms and conditions.

In accordance with the subject invention, ASD has been determined to be of infectious origins associated with any one or more of a variety of infectious agents playing a role. The infections can be congenital in nature resulting from, for example, maternal infections. These infections trigger inflammatory changes including the generation of reactive species and other mediators. These mediators can cause damage to the DNA of the subject's brain, which then produces organic changes in the brain. Both inflammation and DNA damage then induce disruption of the immune system. Further, inflammation causes folate deficiency, which participates in the brain malfunction, all of which leads to ASD.

In many instances, the sequence of events in ASD starts with an infection that triggers the chain of events finally resulting in ASD. The infection may be, for example, passed from a pregnant mother to an unborn fetus.

Specifically described herein is an infectious etiology wherein, first, a fetus is exposed to a pathogen, such as a virus or bacteria, or the fetus is exposed to maternal factors associated with the mother having been exposed to a pathogen. This exposure of the fetus to a pathogen and/or to deleterious immune system factors, induces inflammation and/or other deleterious immune responses in the fetus, which then elicit oxidative stress and/or pro-inflammatory factors that can cause DNA alterations, folate deficiency and/or damage to the gut-brain axis in the fetus or newborn child. These alterations cause abnormal brain development in the fetus and/or child and ultimately result in ASD. Further, these physiological changes compromise the immune system of the subject such that the subject cannot effectively fight the persistent infection, and the cycle repeats itself causing progression and perpetuation of autistic symptoms and ASD-related disorders.

ASD is not just triggered and promoted by these events/changes, but also, because they are interconnected, these factors have a “circularly closed” character that perpetuates the condition. Thus, the initial infection or infections, triggering the events leading to ASD, through a number of alterations, perpetuate ASD because of the inability of the subject's compromised immune system to effectively fight the infection.

The process of ASD symptom development is “continuous” by nature because the disrupted immune system is not capable of eliminating the infection, which additionally “feeds” the ASD process.

The present invention seeks to break the cycle by reducing risk factors that contribute to continued inflammation and resulting neurodegradation.

### Selected Definitions

The terms “autism spectrum disorder” and “ASD” are used in this disclosure to refer to a spectrum of disorders characterized by abnormalities of social interactions and communication, as well as restricted interests and repetitive behavior. This spectrum includes, but is not limited to, autistic disorder (autism), Asperger's syndrome, childhood disintegrative disorder, atypical autism or pervasive developmental disorder not otherwise specified (PPD-NOS), as well as Rett syndrome and tuberous sclerosis.

As used herein, “treating” or “treatment” means the eradicating, improving, reducing, ameliorating or reversing of at least one sign or symptom of a condition or disorder. Treatment can include, but does not require, a complete cure of the condition or disorder, meaning treatment can also include partial eradication, improvement, reduction, amelioration or reversal. Treatment can also include delaying, forestalling and/or inhibiting the progression of a condition or disorder to a more severe condition or disorder.

Signs and/or symptoms of ASD encompass those of ASD or of any comorbidity of ASD. Signs and symptoms associated with ASD include, but are not limited to irritability; hyperactivity; inattention; abnormalities in speech, verbal, communication, and language skills; repetitive behavior, including stereotypy, compulsive behavior, sameness (resistance to change), and ritualistic behavior; obsessive focus on certain topics and/or objects; inability to make eye contact; abnormalities in social interactions and/or understanding of others' feelings; anger issues and/or emotional outbursts; self-injury; and others.

Comorbidities of ASD include, but are not limited to, anxiety; attention deficit disorder; brain inflammation; viral infections; clinical depression; Tourette syndrome; Fragile X syndrome; obsessive-compulsive disorder; bipolar disorder; learning disabilities; sensory disorders; developmental coordination disorder; disorders of the immune system and/or gastrointestinal system, including candidiasis; seizures and/or epilepsy; sleep disorders; increased risk of cancer; and others.

The terms “therapeutically effective” amount or dose, “effective amount,” and “effective dose” are used in this disclosure to refer to an amount of a method, compound or composition that, when administered to a subject, is capable of providing a desired therapeutic effect or a desired level or treatment. The actual amount of the method, compound or composition will vary depending on a number of factors including, but not limited to, the particular disorder being treated, the severity of the disorder, the size and health of the patient, and the route of administration. A skilled medical practitioner can readily determine the appropriate amount using methods known in the medical arts.

A plant “extract,” as used herein, refers to the material resulting from exposing a plant part to a solvent and removing the solvent, or from using various chemical, immunological, biochemical or physical procedures known to those of skill in the art, including but not limited to, precipitation, centrifugation, filtering, column chromatography, and detergent lysis. Plant material can include roots, stems, leaves, flowers, or parts thereof.

As used herein, the term “probiotic” refers to microorganisms, which, when administered in adequate amounts, confer a health benefit on the host. The probiotics may be available in foods and dietary supplements (for example through capsules, tablets, and powders). Non-limiting examples of foods containing probiotics include dairy products such as yogurt, fermented and unfermented milk, smoothies, butter, cream, hummus, kombucha, salad dressing, miso, tempeh, natto, nutrition bars, and some juices and soy beverages. In preferred embodiments, the microorganisms are live.

The terms “natural” and “naturally-derived,” as used in the context of a chemical compound or substance is a material that is found in nature, meaning that it is produced from earth processes or by a living organism. A natural product can be isolated or purified from its natural source of origin and utilized in, or incorporated into, a variety of applications, including foods, beverages, cosmetics, and supplements. A natural product can also be produced in a lab by chemical synthesis, provided no artificial components or ingredients (i.e., synthetic ingredients that cannot be found naturally as a product of the earth or a living organism) are added.

The terms “isolated” or “purified,” when used in connection with biological or natural materials such as nucleic acid molecules, polynucleotides, polypeptides, proteins, organic compounds, such as small molecules, microorganism cells/strains, or host cells, means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

The description herein of any aspect or embodiment of the invention using terms such as “comprising,” “having,” “including,” or “containing” with reference to an element or elements is intended to provide support for a similar aspect or embodiment of the invention that “consists of,” “consists essentially of,” or “substantially comprises” that particular element or elements, unless otherwise stated or clearly contradicted by context (e.g., a composition described herein as comprising a particular element should be understood as also describing a composition consisting, or consisting essentially, of that element, unless otherwise stated or clearly contradicted by context).

The term “consisting essentially of,” as used herein, limits the scope of the ingredients and steps to the specified materials or steps and those that do not materially affect the basic and novel characteristic(s) of the present invention.

Methods for Reducing the Likelihood of Developing ASD and/or Improving the Quality of Life in ASD Subjects

In certain embodiments, the present invention provides a multi-step method for treating a subject with ASD. The method can comprise (1) testing a subject for and/or diagnosing the subject with ASD and/or a risk marker of ASD, as identified herein; (2) analyzing the test results and determining a course of treatment based thereon; and (3) administering the course of treatment to the subject.

In some embodiments, the method can further comprise intermittently assessing the effectiveness of the treatment; and, based on the intermittent assessment, adjusting the treatment as needed.

In one embodiment, the method can be used to treat and/or reduce the severity of symptoms of ASD, including behavioral, mental, emotional and/or physiological symptoms. The subject can be any human diagnosed with ASD. In one embodiment, the human subject is a child or adolescent, for example, 16 years of age or younger.

In certain embodiments, the present invention provides a multi-step method for preventing and/or reducing the severity of in utero development of ASD in a fetal or neonatal subject. The method can comprise (1) testing a woman who is pregnant, or who plans to become pregnant, for ASD risk markers as identified herein; (2) analyzing the test results and determining a course of treatment for the woman based thereon; and (3) administering the course of treatment to the woman.

In some embodiments, the method can further comprise intermittently assessing the effectiveness of the treatment; and, based on the intermittent assessment, adjusting the treatment for the mother as needed throughout the pregnancy and afterwards (e.g., throughout the time that the child is nursing).

In certain embodiments, the woman has a personal or family history of ASD or other neurodevelopmental conditions and/or is determined to have a genetic marker associated with ASD. In certain embodiments, the woman has previously given birth to one or more children diagnosed with ASD.

In one embodiment, the method can be used to reduce the likelihood that a woman will give birth to a child with ASD, and/or reduce the severity of symptoms that occur in a child born with ASD, including behavioral, mental, emotional and/or physiological symptoms.

In one embodiment, the “subject” according to the present invention is a child diagnosed with ASD or who exhibits signs and symptoms thereof. In another embodiment, the subject is a child, fetus or expectant mother who has a compromised immune system, for example, due to genetics, illness, or because the subject previously contracted or was exposed to a viral or microbial infection. The subject may presently be exhibiting signs of infection, or the subject may be asymptomatic because, for example, contraction of the virus occurred at some time in the past, e.g., in utero.

Infection or exposure according to the subject disclosure typically occurs early on in a subject's development, either through vertical transmission (i.e., from mother to embryo, fetus, or infant during pregnancy or childbirth), direct transmission (i.e., contact from another infected subject), or indirect transmission (e.g., through a vector).

### Testing

In one embodiment, the method of the subject invention comprises testing a subject for, and/or diagnosing the subject with, ASD and/or a risk marker. According to the subject invention, testing can comprise, for example, conducting a comprehensive blood test to determine the presence and extent of risk markers, either in the subject, or in a woman who is pregnant or plans to become pregnant.

Testing can include CARS™ (Childhood Autism Rating Scale) testing, gastrointestinal system testing (e.g., stool sampling) for gut microbiome analysis, reviewing personal and family medical history, and in the case of pathogenic infections that cause, e.g., external lesions, sampling, culturing and polynucleotide sequencing.

The risk markers may be, for example, a viral infection or an immune system response to a viral infection. In one embodiment, the method can comprise testing the subject for signs of poor immune health by, for example, testing the subject for immune markers such as T-cells and natural killer (NK) cells. The risk markers may also be genetic markers and/or family history. The testing can be performed using known testing methods.

In another embodiment, the subject invention comprises testing a woman who is pregnant, or who plans to become pregnant, for the ASD risk markers as identified herein. As with the risk markers for the child, the risk markers for which the woman can be tested include, but are not limited to, a pathogenic infection and/or an immune system response that is consistent with a pathogenic infection. Genetic testing and/or family history evaluation can also be done.

In certain embodiments, the risk markers according to the subject invention can include signs of poor immune health, for example, immune markers such as T-cells and NK cells. Other exemplary risk markers can include, for example, white blood cell counts, red blood cell counts, ESR, CRP, immunoglobulin E, A, and G levels, thyroid stimulating hormone (TSH) counts, T3, T4, insulin, glucose, mineralocorticoids, cholesterol, HDL and LDL levels.

In certain embodiments, the subject has previously contracted an infection, currently has an infection, or has been exposed to one or more infectious agents. Thus, the risk markers can be antibodies indicating a previous or current infection or exposure to an infection agent selected from one or more of cytomegalovirus (CMV), Epstein-Barr virus (EBV), rubella virus, measles virus, herpes simplex virus (HSV) type 1 or 2, herpes zoster, varicella-zoster virus (VZV), HHV6, HHV7, other herpes family viruses, Mycoplasma, Chlamydia, Helicobacter, Toxoplasma, or any other chronic, congenital, persistent, latent, dormant, acute and/or subacute infection. In preferred embodiments, the infection is due to a TORCH infection (Toxoplasmosis, Other (Syphilis, Parvovirus B19, HIV, HBV, and Varicella zoster), Rubella, CMV and/or HSV).

In one embodiment, the expectant mother, the fetus, neonate and/or child has an abnormal microbiota, e.g., Clostridial overgrowth, Staphylococcus overgrowth, Candida overgrowth, Aspergillus overgrowth, SIBO, SIFO, or any biofilm-related disease.

In one embodiment, the expectant mother and/or child has an autoimmune disorder, such as, for example, diabetes, psoriasis, lupus, Cron's, vitiligo, Hashimoto's hypothyroidism, Addison's disease, and others.

### Treatment

After determining the identity and extent of risk factors present in the expectant mother, fetus, neonate and/or child, the methods of the present invention can further comprise treating the expectant mother, fetus, neonate and/or child to reduce the occurrence and/or severity of ASD. Preferably, treatment comprises a combination of prescription medications and/or over-the-counter medications with a unique blend of supplement formulations that address various risk factors, including, e.g., infection, poor digestive and mucosal health, poor immune health, metabolic conditions, endocrine disruption and inflammation.

In view of the ASD cycle, treatment according to the subject invention focuses on interrupting the cycle at various points. The interruption of the cycle may be, for example, treating a source of inflammation, or treating an existing condition such as an autoimmune condition. Thus, exemplary medications such as NSAIDS, insulin, omega-3 fatty acids, statins, antihistamines, and hormone replacements (e.g., levothyroxine) can be administered to the subject and/or an expectant mother who has such a condition.

The interruption of the cycle may also be, for example, in the form of treating an infection in the mother, the fetus, or a child. Treatment for the infection can include, for example, one or more of administering antibiotics, antifungal and/or antiviral agents, and boosting the immune system. Medications can be selected based upon the results of the testing, for example, antibody testing and/or GI testing.

In certain embodiments, antibiotics can include, for example, penicillins (such as penicillin G, penicillin V, ampicillin, amoxicillin, bacampicillin, carbenicillin, carbenicillin indanyl, ticarcillin, azlocillin, mezlocillin, methicillin, piperacillin, and the like), tetracyclines (such as chlortetracycline, oxytetracycline, methacycline, doxycycline, minocycline and the like), cephalosporins (such as cefadroxil, cephalexin, cephradine, cephalothin, cephapirin, cefazolin, cefaclor, cefamandole, cefonicid, cefoxitin, cefotetan, cefuroxime, cefuroxime axetil, cefinetazole, cefprozil, loracarbef, ceforanide, cefepime, cefoperazone, cefotaxime, ceftizoxime, ceftriaxone, ceftazidime, cefixime, cefpodoxime, ceftibuten, and the like), fluoroquinolones (e.g., levofloxacin), quinolones (such as nalidixic acid, cinoxacin, ciprofloxacin and norfloxacin and the like), lincomycins (e.g., clindamycin), macrolides (e.g., erythromycin, azithromycin), sulfones (e.g., dapsone), sulfonamides (e.g., sulfanilamide, sulfadiazine, sulfamethoxazole, sulfisoxazole, sulfacetamide, bactrim), lipopeptides (e.g., daptomycin), polypeptides (e.g., bacitracin), glycopeptides (e.g., vancomycin), aminoglycosides (e.g., streptomycin, gentamicin, tobramycin, amikacin, netilmicin, kanamycin, and the like), nitoimidazoles (e.g., metronidazole) and/or carbapenems (e.g., thienamycin).

Antivirals can be, for example, valacyclovir, acyclovir, famciclovir, ganciclovir, valganciclovir, ribavirin, brivudin, cidofovir, fomivirsen, foscarnet, penciclovir, vidarabine and others used to treat chronic, congenital, persistent, latent, dormant, acute and/or subacute viral infections.

Antifungals can be, for example, clotrimazole, econazole, miconazole, terbinafine, fluconazole, ketoconazole, nystatin and amphotericin.

The immune system can be boosted by, for example, the administration of immunomodulatory compositions, the use of anti-oxidants, and/or the administration of probiotics.

In certain specific embodiments, the step of administering an antiviral, antifungal or antibacterial treatment comprises administering a therapeutically-effective dose of a prescription medication, although naturally-derived treatments are also envisioned.

In one specific embodiment, treatment comprises administering an antiviral medication selected from the group consisting of valacyclovir (also known as Valtrex), acyclovir (also known as Zovirax), ganciclovir (also known as, e.g., Cytovene) and famciclovir (also known as Famvir).

Each of these commercially available drugs was originally designed for treatment of adults infected with herpes family viruses. As a preferred example, the antiviral treatment is valacyclovir. Valacyclovir can be administered to the subject once or twice a day, at up to 500 mg per dose, up to 250 mg per dose or up to 125 mg per dose. Proper dosage of the antiviral treatment is determined by a skilled physician based on the individual receiving treatment, with factors such as age and symptoms considered.

Valacyclovir, most commonly administered for treating and suppressing genital herpes, can be given to patients with ASD to suppress the herpes virus, if present, and decrease inflammation and neurological dysfunction it causes. Valacyclovir can also be useful in managing and/or treating herpes zoster (shingles), cytomegalovirus, EBV, mononucleosis, and herpes B.

Valacyclovir is the prodrug of acyclovir, another common antiviral treatment. Acyclovir differs from valacyclovir in that it typically requires more frequent dosage.

Children with ASD may show improvements in any aspect of their spectrum of symptoms in response to treatment with these antiviral medications, including but not limited to improvement in behavioral performance.

In embodiments of the present invention, administration of the antiviral, antifungal or antibacterial medication occurs daily for several months or longer. Administration can include any known method of drug administration, including, but not limited to, oral, nasal, cutaneous (e.g., applying it as a cream), or intravenous administration. In preferred embodiments, administration is performed orally once or twice daily.

The medication can be administered in cycles, for example, four months on with two months off, or three weeks on with one week off. Cycles can be repeated multiple times, for example three times, or as many times as deemed necessary by a skilled physician.

In preferred embodiments, at the start of treatment, the antiviral, antifungal or antibacterial treatment is administered alongside one or more proprietary supplement formulations described herein, which are specifically designed to, for example, decrease infections and inflammation, while also regulating mitochondrial activity.

In some embodiments, the antiviral, antifungal or antibacterial treatment is administered to the subject on its own for one week, two weeks or three weeks, to determine whether the subject will experience any adverse side-effects from the medication. Then, the supplement composition(s) can be introduced into the treatment.

In one embodiment, one or more supplement compositions are ingested by the subject once, twice, or three times per day, determined on a subject-by-subject basis by a skilled physician. Factors to be considered when determining the number of doses to administer include the age of the individual receiving treatment and the severity of his/her symptoms. Supplement formulations that are administered are determined based on testing results and risk markers identified prior to treatment.

The concomitant administration of antiviral and supplement composition can be continued for one or more consecutive cycles lasting, for example, 21 days or longer. A cycle can be repeated as many times as necessary without breaking in between, for example, 3 times or more.

In some embodiments, treatment further comprises prescribing a specified diet regimen to an expectant mother or a child, wherein the diet has therapeutic effects toward the prevention and/or improvement of ASD symptoms. In one embodiment, the diet is a low-sugar or low-carbohydrate diet, such as the “Keto Diet” or “Atkins® Diet.” In another embodiment, the subject's diet can be supplemented with sources of protein, such as protein powders, beef protein, and beef liver protein.

In some embodiments, treatment further comprises sleep therapeutics, wherein the subject is administered a sleep aide such as L-threonine, melatonin, or Benadryl.

Treatment is intermittently adjusted based on behavioral and medical improvements until a maintenance “supplements only” treatment is achieved. Assessment can include, for example, periodic blood work throughout the treatment time period, for example every week, every month, or every two months. The blood testing can not only be used to monitor the level of, for example, risk markers such as viral antibodies and/or viral load in a subject, but also monitor how the subject's liver, kidneys, blood cells and other body functions are operating. These tests can serve as precautionary safeguards, as harmful side effects from these treatments are rare or even nonexistent.

In some embodiments, the methods disclosed herein can also include measuring a baseline of behavioral performance and/or overall health prior to treatment of the subject according to the subject methods, and/or measuring the behavioral performance and/or overall health after treatment. The methods can include comparing the behavioral performance and/or overall health prior to and after treatment is administered to the subject, and the comparison can be used to determine if and to what extent the behavioral performance and/or overall health in the subject is improved, or if adjustments should be made to the treatment given.

As used herein, the phrase “improvement in behavioral performance” refers to reduction in the severity or frequency, to whatever extent, of one or more of the behavioral disorders, symptoms and/or abnormalities expressed by an individual suffering from ASD, or a pathological condition having behavioral symptoms similar to those of ASD. The improvement is either observed by the individual taking the treatment themselves or by another person (medical professional or otherwise).

In the method disclosed herein, behavioral performance can be measured and evaluated using various parameters and methods. For example, behavioral tests can be conducted to determine the presence and/or extent of restricted repetitive behavior and/or stereotyped behavior patterns of the subject under test. In some embodiments, the Autism Behavior Checklist (ABC), Autism diagnostic Interview-Revised (ADI-R), childhood autism Rating Scale (CARS), and/or Pre-Linguistic Autism Diagnostic Observation Schedule (PL-ADOS) is used for the behavioral test. The behavioral test can include, but is not limited to, detecting the presence and/or extent of (1) preoccupation with one or more stereotyped and restricted patterns of interest that is abnormal in either intensity or focus; (2) inflexible adherence to specific, nonfunctional routines or rituals; (3) stereotyped and repetitive motor mannerisms (such as hand flapping, finger flapping etc.); and/or (4) persistent preoccupation with parts of objects. Non-limiting examples of behavior that can be included in a behavioral test and suggest a need for improving behavioral performance in the subject under the test include:
(a) Sensory behaviors: poor use of visual discrimination when learning; seems not to hear, so that a hearing loss is suspected; sometimes shows no “startle response” to loud noise; sometimes painful stimuli such as bruises, cuts, and injections evoke no reaction; often will not blink when bright light is directed toward eyes; covers ears at many sounds, squints, frowns, or covers eyes when in the presence of natural light; frequently has no visual reaction to a “new” person; stares into space for long periods of time;
(b) Relating behaviors: frequently does not attend to social/environmental stimuli; has no social smile; does not reach out when reached for; non-responsive to others' facial expressions/feelings; actively avoids eye contact; resists being touched or held; is flaccid when held in arms; is stiff and hard to hold; does not imitate other children at play; has not developed any friendships; is often frightened or very anxious; “looks through” people;
(c) Body and object use behaviors: whirls self for long periods of time; does not use toys appropriately; insists on keeping certain objects with him/her; rocks self for long periods of time; frequently lunges and darts; flaps hands; walks on toes; hurts self by banging head, biting hand, etc.; twirls, spins, and bangs objects frequently; will feel, smell, and/or taste objects in the environment; performs complicated “rituals” such as lining things up; is very destructive; and
(d) Language behaviors: does not follow simple commands given once; has pronoun reversal; speech is atonal; does not respond to own name when called out among others; seldom says “yes” or “I”; does not follow simple commands involving prepositions; uses gestures to get desired objects; repeats phrases over and over; cannot point to more than five named objects; uses 0-5 spontaneous words per day to communicate wants and needs; repeats sounds or words over and over; echoes questions or statements made by others; uses at least 15 but less than 30 spontaneous phrases daily to communicate; learns a simple task but “forgets” quickly; has strong reactions to changes in routine/environment; has “special abilities” in one area of development, which seems to rule out mental retardation; has severe temper tantrums and/or frequent minor tantrums; hurts others by biting, hitting, kicking, etc.; does not wait for needs to be met; has difficulties with toileting; does not dress self without frequent help; is frequently unaware of surroundings and may be oblivious to dangerous situations; prefers to manipulate and be occupied with inanimate things; and a developmental delay was identified at or before 30 months of age.

One of ordinary skill in the art would appreciate that the attending physician would know how to identify a subject in need of treatment disclosed herein.

### Supplement Compositions

In certain embodiments, the present invention provides supplement formulations with specified functions, which are useful according to the methods described herein.

In one embodiment, the supplement compositions help support the immune system of a subject with a compromised immune system, for example, a subject diagnosed with ASD and/or a viral infection. As used herein, the term “support” can include boosting, improving, enhancing, and/or maintaining the proper functioning of a body system, for example, those involved in the immune system.

Immune support can include support for the cells, tissues, and organs that contribute to proper functioning of the immune system, for example, the lymphatic system, spleen, bone marrow, or any other system involved in production of entities (e.g., antibodies, lymphocytes, red blood cells, white blood cells, platelets) that ward off foreign substances (e.g., inoculants such as bacteria, viruses, parasites, and fungi) from the body's normal and healthy tissues. Immune support can further include support for parts of the body that aid in preventing and healing from injury, inflammation, cancer, or other non-infectious diseases, ailments, or conditions.

In some aspects, the supplement compositions can also help support other body systems that are known to be interrelated with immune health, such as the circulatory, endocrine, urinary, muscular, respiratory, skeletal, central nervous, digestive, integumentary, and reproductive systems. In other words, the subject compositions can be effective in supporting more than one body system, particularly where the health of one body system promotes the health of another.

In certain specific embodiments, supplement compositions are formulated for (1) suppression of low-grade inflammation and reactive molecules; (2) recovery of normal mucosa and mucosal immunity; (3) establishment of healthy microbiota and immunity restoration; (4) treatment and modulation of metabolic and endocrine abnormalities; (5) restoration of normal neural functions and enhancement of neurogenesis; and/or (6) recovery and “fine-tuning” of emotional and cognitive spheres. (Table 1).

Preferably, the supplement formulations are administered in a specific cascading order from F1 to F6, ending in a cumulative administration of all six different formulations; however, in some embodiments, different formulations can be administered individually or in any combination or order.

TABLE 1
Supplement Compositions

| Formula | Components |
|---|---|
| F1 - Antioxidant and | In preferred embodiments, F1 comprises therapeutically effective |
| Inflammation Support | amounts of one or more of: curcumin, N-acetylcysteine, |
| | resveratrol, zinc and piperine. |
| F2 - GI Health and | In preferred embodiments, F2 comprises therapeutically effective |
| Mucosal Immunity | amounts of one or more of: monolaurin, L-lysine, caprylic acid, |
| Support | pentadecanoic acid, and vitamin D. |
| F3 - GI and General | In preferred embodiments, F3 comprises therapeutically effective |
| Health Probiotic | amounts of one or more of: Lactobacilli, Bifidobacteria, Bacilli, |
| | and banana powder. |
| F4 - Metabolic and | In preferred embodiments, F4 comprises therapeutically effective |
| Endocrine Health | amounts of one or more of: berberine, diindolymethane (DIM), |
| Support | cordyceps, magnesium, and microelements (potassium iodine, |
| | selenium and/or chromium). |
| F5 - Neurogenesis | In preferred embodiments, F5 comprises therapeutically effective |
| Support | amounts of one or more of: taurine, electrolytes, honokiol, B |
| | vitamins and piperine. |
| F6 - Mood and Anxiety | In preferred embodiments, F6 comprises therapeutically effective |
| Support | amounts of one or more of: ashwagandha, L-theanine, saffron, |
| | Gingko biloba extract, and piperine. |

### Formulation and Delivery of Supplement Compositions

In a preferred embodiment, the supplement composition of the present invention is formulated so that it can be delivered to a subject orally. In particular, the compositions are formulated as an orally-consumable product.

Orally-consumable products according to the subject invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally-consumable products (e.g., active ingredients such as extracts, nutrients, supplements, or pharmaceutical products) during their production, treatment or processing and intended to be introduced into the human or animal oral cavity.

Orally-consumable products can also include substances intended to be swallowed by humans or animals and then digested in an unmodified, prepared or processed state. These include casings, coatings or other encapsulations that are intended also to be swallowed together with the product or for which swallowing is to be anticipated.

Preferably, the orally-consumable product according to the invention is formulated as a composition to be consumed for nutrition or pleasure. These particularly include baked goods (e.g., bread, dry biscuits, cake, cookies, brownies and other pastries), sweets and candies (e.g., chocolates, chocolate bar products, other bar products, gummies, fruit leathers, jelly beans, coated tablets, hard candies, toffees and caramels, and chewing gum), non-alcoholic beverages (e.g., cocoa, coffee, green tea, black tea, herbal teas, lemonades, isotonic beverages, soft drinks, nectars, fruit and vegetable juices, and fruit or vegetable juice preparations), instant beverages (e.g., instant cocoa beverages, instant tea beverages, instant smoothies, instant milkshakes and instant coffee beverages), meat products (e.g., cold cuts, fresh or raw sausage preparations, seasoned oder, marinated fresh meat or salted meat products), eggs or egg products (e.g., dried whole egg, egg whites, and egg yolks), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, ice cream, sherbet, whey, butter, buttermilk, and partly or wholly hydrolyzed products containing milk proteins), products produced from nuts (e.g., nut milks, nut butters, nut flours or powders), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sorbets, fruit smoothies, fruit sauces, and fruit fillings), vegetable preparations (e.g., ketchup, sauces, dried vegetables, deep-freeze vegetables, pre-cooked vegetables, and boiled vegetables), snack articles (e.g., chips, crisps, pretzels, biscuits, crackers and nuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening beverages or other food). The present compositions may also serve as semi-finished products for the production of other compositions intended for nutrition or pleasure.

Preferably, the present composition is delivered by an orally-consumable product that appeals to children, for example, in the form of a sweet treat or snack. In a preferred embodiment, the composition is delivered in the form of a chocolate bar, a gummy or a jelly.

When formulated as a chocolate bar, the chocolate can be dark chocolate, light chocolate, milk chocolate, white chocolate or a mixture thereof. Optionally, caramel, nut butters, fruit or fruit fillings, coconut, sprinkles, chips (chocolate or other), candies, or any of a variety of other flavorings or food products can be added to the bar to enhance the taste and appeal thereof.

Preferably, the chocolate bars are packaged in blister packs, or in larger, multi-dose break-apart bars with individual sections constituting one dose. The chocolate bars can also be packaged as individually wrapped single-dose bars.

In certain embodiments, the composition is formulated as a gummy or a jelly, or some other form of gelatinous or chewy candy. For example, the composition can be formulated as a gummy bear, gummy worm, or other well-known gummy candy, a fruit snack, a fruit tape, a fruit leather, a jelly bean or a taffy. A single dose of the supplement composition can be included in one piece of the candy, or divided between a pre-determined number of candies, for example from 2 to 5 pieces.

The composition of the subject invention can also be present in the form of capsules, tablets (uncoated and coated tablets, e.g., gastro-resistant coatings), coated tablets, granules, pellets, solid-substance mixtures, dispersions in liquid phases, as emulsions, powders, solutions, pastes or other swallowable or chewable preparations, or as a dietary supplement.

For oral administration, tablets or capsules can be prepared by conventional means with acceptable excipients such as binding agents, fillers, lubricants, disintegrants, or wetting agents. The tablets can be coated, if desired. Liquid preparations for oral administration can take the form of, for example, solutions, syrups, or suspension, or they can be presented as a dry product for constitution with saline or other suitable liquid vehicle before use.

The compositions described herein can also contain acceptable additives as will be understood by one skilled in the art, depending on the particular form of the delivery method. Non-limiting examples of such additives include suspending agents, emulsifying agents, non-aqueous vehicles, preservatives, buffer salts, flavoring, coloring, and sweetening agents as appropriate. Non-limiting examples of specific additives include: gelatin, glycerin, water, beeswax, lecithin, cocoa, caramel, titanium dioxide, and carmine. Preparations for oral administration also can be suitably formulated to give controlled release of the active ingredients.

In some cases, the composition provided herein can contain an acceptable carrier for administration to a human subject or other mammal including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers appropriate to specific routes of administration.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Comprehensive Blood Test

The first step according to embodiments of the subject invention involves testing a subject for ASD and/or risk markers for ASD. The following is an example of a comprehensive blood test that can be performed to determine the extent of risk factors exhibited in a child or an expectant mother.

TABLE 2
Comprehensive blood test

| | | | |
|---|---|---|---|
| Complete | Lymphocyte Subset | Antibodies G and M | Inflammation |
| blood count | Panel: | to several infections: | markers: |
| and | Absolute lymphocytes | HSV | Interleukin-6 |
| differential | % CD 3 (Mature T-Cells) | EBV | Tumor Necrosis Factor TNF |
| | Absolute CD3+ cells | CMB | C-reactive protein |
| | % CD4 (Helper Cells | Rubella virus | Ferritin |
| | Absolute CD4+ cells | Chlamydia | Erythrocyte |
| | % CD8 (Suppressor T-Cells) | Helicobacter | sedimentation rate (in |
| | Absolute CD8+ cells | Mycoplasma | case it is not included |
| | Helper/Suppressor ratio | Toxoplasma | with the complete blood |
| | | Anti-streptolysin O | count |

### Example 2—Supplement Formula F1

In preferred embodiments, supplement formula F1 comprises therapeutically effective amounts of one or more of: curcumin, N-acetylcysteine, resveratrol, zinc and piperine. F1 can be useful for, e.g., antioxidant and inflammation support.

TABLE 3
Supplement formula F1

| Compound | Amount | Mechanism of Action |
|---|---|---|
| Curcumin | 100-500 mg, | A potent antioxidant that can neutralize free radicals |
| | 150-450 mg, | both by blocking the action of free radicals and by |
| | 200-400 mg, | stimulating the action of other antioxidants. Also, it |
| | or | aids in the management of inflammatory conditions, |
| | 250-300 mg | metabolic syndrome, arthritis, anxiety, and |
| | | hyperlipidemia. |
| N-acetylcysteine | 25-200 mg, | A compound essential for making the powerful |
| | 50-150 mg, | antioxidant glutathione. It can improve immune |
| | or | function and inactivate primary toxicants or reactive |
| | 75-100 mg | electrophiles arising as metabolites or lipid |
| | | peroxidation products. It may improve the brain |
| | | function and mental health conditions. |
| Resveratrol | 25-250 mg, | A phenolic compound with antioxidant and anti- |
| | 50-200 mg, | inflammatory properties and is thought to protect |
| | or | against diseases like cancer, diabetes, Alzheimer's |
| | 75-150 mg | disease, and other neurological pathologies. Also, |
| | | resveratrol has antibacterial and antifungal properties |
| | | that help treat infections of the urinary and digestive |
| | | tracts. |
| Zinc | 1 to 20 mg, | Zinc is essential for many body functions. It is a part |
| | 2 to 10 mg, | of many proteins including critical enzymes. It is |
| | or | required for numerous processes like molecular |
| | 3-5 mg | signaling for immune cells and many transcription |
| | | factors involved in gene expression of inflammatory |
| | | cytokines and adhesion molecules. |
| Piperine | 1 to 20 mg, | Piperine's main function in this composition is the |
| | 2 to 10 mg, | increase of nutrient absorption. Also, it may alleviate |
| | or | inflammation, support healthy metabolism and |
| | 3-5 mg | healthy brain function. |

### Example 3—Supplement Formula F2

In preferred embodiments, supplement formula F2 comprises therapeutically effective amounts of one or more of: monolaurin, L-lysine, caprylic acid, pentadecanoic acid, and vitamin D. F2 can be useful for, e.g., GI health and mucosal immunity support.

TABLE 4
Supplement formula F2

| Compound | Amount | Mechanism of Action |
|---|---|---|
| Monolaurin | 25-250 mg, | Monolaurin is used for preventing and treating viral |
| | 50-200 mg, | infections including herpes, shingles, and other |
| | or | infections. It is also used to treat chronic fatigue |
| | 75-150 mg | syndrome (CFS), asthenic syndrome and to improve |
| | | the immune system functioning. |
| L-lysine | 50-300 mg, | It helps the body to absorb calcium, and it plays an |
| | 75-250 mg, | important role in the formation of collagen, a |
| | or | substance important for bones and connective tissues |
| | 100-200 mg | including skin, mucosal membranes, tendons, and |
| | | cartilage. |
| Caprylic acid | 25-250 mg, | It helps treat inflammatory digestive disorders. Some |
| | 50-200 mg, | research suggests it can be useful in managing yeast |
| | or | infections, skin conditions, digestive disorders, and |
| | 75-150 mg | high cholesterol. |
| Pentadecanoic | 25-200 mg, | Highly effective antioxidant and inflammation |
| acid | 50-150 mg, | suppressant (more effective than Omega-3). It can |
| | or | improve gut health |
| | 75-100 mg | |
| Vitamin D | 25-200 mcg, | Vitamin D reduces a likelihood of severe morbidities |
| | 50-150 mcg, | by strengthening the general and mucosal immune |
| | or | systems. Also, it reduces the risks of metabolic and |
| | 75-100 mcg | neurologic disorders. |

### Example 4—Supplement Formula 3

In preferred embodiments, supplement composition F3 comprises therapeutically effective amounts of one or more of: Lactobacilli, Bifidobacteria, Bacilli, and banana powder. F3 can be useful for, e.g., GI health and overall health.

TABLE 5
Supplement formula F3

| Main | | |
|---|---|---|
| compounds | Details | Mechanism of Action |
| 100 mg of | L. acidophilus, | These probiotic bacteria inhibit or |
| Lactobacillus | L. plantarum, | decrease the growth of harmful |
| strains: a | L. bulgaricus, | microorganisms in the gut by |
| composition | L. paracasei, L. casei, | producing lactic acid. |
| with 30 billion | L. brevis, L. rhamnosus, | Also, most importantly, these |
| CFUs/capsule | L. salivaris, L. gasseri, | probiotics showed efficacy in |
| | L. reuteri. | improving psychiatric disorder-related |
| | | behaviors including anxiety, |
| | | depression, autism spectrum disorder |
| | | (ASD), obsessive-compulsive |
| | | disorder, and memory abilities, |
| | | including spatial and non-spatial |
| | | memory |
| 50 mg of | B. lactis, B. breve, | Bifidobacteria regulate host immune |
| Bifidobacterium | B. bifidum, B. longum. | response and positively influence |
| strains: a | | central nervous system (CNS) |
| composition | | function through direct and indirect |
| with 15 billion | | mechanisms. |
| CFUs/capsule | | Also, they can positively influence |
| | | hypothalamic-pituitary-adrenal |
| | | (HPA) axis by altering corticosteroid |
| | | (CORT) and/or adrenocorticotropic |
| | | hormone (ACTH) levels. |
| | | As well, it was shown to reduce the |
| | | lipid peroxidation and improve some |
| | | metabolic functions of the body |
| 50 mg of | Bac. coagulans and | These probiotic bacillus can inhibit the |
| Bacillus stains: a | Bac. natto | growth of harmful microorganisms, |
| composition | | induce an anti-inflammatory state, |
| with 5 billion | | modulate immune response, and |
| CFUs/capsule | | produce surfactin and nattokinase in |
| | | the gut. |
| Banana powder- | A source of prebiotics | Acts as a powerful prebiotic and a |
| 300 mg and | and postbiotics | powerful source of nutrients to |
| supernatant | | enhance adhesion and propagation of |
| powder-100 mg | | probiotic bacteria. |

### Example 5—Supplement Formula 4

In preferred embodiments, supplement composition F4 comprises therapeutically effective amounts of one or more of: berberine, diindolymethane (DIM), Cordyceps, magnesium, and microelements (potassium iodine, selenium and/or chromium). F4 can be useful for, e.g., metabolic and endocrine health support.

TABLE 6
Supplement formula F4

| Compound | Amount | Mechanism of Action |
|---|---|---|
| Berberine | 50-300 mg, | Berberine can effectively regulate |
| | 75-250 mg, | blood glucose and blood lipid of |
| | or | patients, alleviate insulin resistance |
| | 100-200 mg | and reduce the level of inflammatory |
| | | response in the body. |
| Diindolyl- | 25-200 mg, | DIM supports estrogen balance by |
| methane (DIM) | 50-150 mg, | increasing beneficial 2-hydroxy |
| | or | estrogens and reducing the unwanted |
| | 75-100 mg | 16-hydroxy variety. Also, DIM has |
| | | been shown to inhibit an enzyme |
| | | called aromatase, which converts |
| | | testosterone to estrogen and may |
| | | correct estrogen imbalances in men. |
| Cordyceps | 50-300 mg, | Cordyceps is an adaptogen that can |
| | 75-250 mg, or | support the adrenal glands and can |
| | 100-200 mg | help adapt to every day's stressors. |
| | | Also, it can improve the body's |
| | | oxygenation and help manage |
| | | metabolic disorders like type 2 |
| | | diabetes |
| Magnesium | 25-200 mg, | Magnesium is essential for the |
| | 50-150 mg, | synthesis of nucleic acids and |
| | or | proteins. Over 300 enzymes are |
| | 75-100 mg | dependent on magnesium. It also acts |
| | | at both the pituitary and adrenal levels |
| | | regulating ACTH and cortisol within |
| | | a normal range. |
| Microelement | a) Potassium iodine: | Iodine is an element that plays an |
| composition | 50-150 mcg | essential role in the functioning of a |
| | b) Selenium: 5-15 | healthy thyroid. |
| | mcg | Selenium contributes to the |
| | c) Chromium: 5-10 | antioxidant defense in the thyroid. |
| | mcg | Chromium helps to increase insulin |
| | | synthesis and positively regulate |
| | | thyroid function. |

### Example 6—Supplement Formula F5

In preferred embodiments, supplement composition F5 comprises therapeutically effective amounts of one or more of: taurine, electrolytes, honokiol, B vitamins and piperine. F5 can be useful for, e.g., neurogenesis support.

TABLE 7
Supplement formula F5

| Compound | Amount | Mechanism of Action |
|---|---|---|
| Taurine | 25-250 mg, | Taurine demonstrates multiple cellular functions including |
| | 50-200 mg, | a central role as a neurotransmitter, as a trophic factor in |
| | or | CNS development, in maintaining the structural integrity of |
| | 75-150 mg | the membrane, in regulating calcium transport and |
| | | homeostasis, as an osmolyte, as a neuromodulator and as a |
| | | neuroprotectant. |
| Electrolyte | 100-500 mg, | Sources of sodium, potassium, phosphate, bicarbonate, |
| mix | 150-450 mg, | sulfate, chloride, calcium and/or magnesium can enhance |
| | 200-400 mg, | hydration and ion transfer. |
| | or | |
| | 250-300 mg | |
| Honokiol | 25-250 mg, | Honokiol is a poly-phenolic compound that exerts |
| | 50-200 mg, | neuroprotective properties through a variety of |
| | or | mechanisms. It has therapeutic potential in anxiety, pain, |
| | 75-150 mg | cerebrovascular injury, epilepsy, and cognitive disorders |
| | | including Alzheimer's disease. |
| B vitamins | 1 to 100 mg, | |
| | 10 to 75 mg, | |
| | or | |
| | 25-50 mg | |
| Piperine | 1 to 20 mg, | Piperine's main function in this composition is the increase |
| | 2 to 10 mg, | of nutrient absorption. Also, it may alleviate inflammation, |
| | or | support healthy metabolism and healthy brain function. |
| | 3-5 mg | |

### Example 7—Supplement Formula F6

In preferred embodiments, supplement formula F6 comprises therapeutically effective amounts of one or more of: ashwagandha, L-theanine, saffron, Gingko biloba extract, and piperine. F6 can be useful for, e.g., mood and anxiety support.

TABLE 8
Supplement formula F6

| Compound | Amount | Mechanism of Action |
|---|---|---|
| Ashwa- | 100 to 400 mg, | It has demonstrated efficacy as an |
| gandha | 150 to 350 mg, | alternative treatment for mild to moderate |
| | or | depression. |
| | 200 to 250 mg | |
| L-theanine | 25-200 mg, | Theanine can significantly reduce |
| | 50-150 mg, | symptoms of anxiety and helps to prevent |
| | or | the brain from producing an anxious |
| | 75-100 mg | response to stressful situations. |
| Saffron | 1 to 100 mg, | It has demonstrated efficacy as an |
| | 5 to 50 mg, | alternative treatment for mild to moderate |
| | or | depression. |
| | 10 to 30 mg | |
| Gingko | 25-250 mg, | Ginkgo biloba is used around the world to |
| biloba | 50-200 mg, | treat a wide variety of ailments including |
| extract | or | memory loss, concentration problems, |
| | 75-150 mg | mental confusion, depression, anxiety, |
| | | dizziness, dementia, eye problems, |
| | | tinnitus, leg pain, and headache. |
| Piperine | 1 to 20 mg, | Piperine's main function in this |
| | 2 to 10 mg, | composition is the increase of nutrient |
| | or | absorption. Also, it may alleviate |
| | 3-5 mg | inflammation, support healthy metabolism |
| | | and healthy brain function. |

1. A method for reducing the likelihood of a subject developing Autism Spectrum Disorder (ASD), the method comprising:
testing the subject for risk markers of ASD, or, if the subject is a fetus of a pregnant woman, testing the pregnant woman for the risk markers, and,
if one or more of the risk markers is detected, treating the subject and/or the pregnant woman with an antibiotic, an antiviral, or an antifungal compound and administering to the subject and/or the pregnant woman one or more adjuvant supplement compositions.

2. The method of claim 1, wherein the risk marker is selected from genetic markers, family history, pathogenic infections, immune system responses to a pathogenic infection, and signs of poor immune health.

3. The method of claim 1, wherein the subject is a child aged 16 years or younger.

4. The method of claim 1, wherein the subject or the pregnant woman has previously contracted an infection, currently has an infection, or is known to have been exposed to one or more infectious agents.

5. The method of claim 4, wherein the subject or the pregnant woman is infected with a TORCH infection.

6. The method of claim 1, wherein the antiviral agent is valacyclovir or ganciclovir.

7. The method, according to claim 1, wherein the one or more supplement compositions are selected from:
F1, which comprises therapeutically-effective amounts of one or more ingredients selected from curcumin, N-acetylcysteine, resveratrol, zinc and piperine;
F2, which comprises therapeutically effective amounts of one or more of: monolaurin, L-lysine, caprylic acid, pentadecanoic acid, and vitamin D;
F3, which comprises therapeutically effective amounts of one or more of: Lactobacilli, Bifidobacteria, Bacilli, and banana powder;
F4, which comprises therapeutically effective amounts of one or more of: berberine, diindolymethane (DIM), Cordyceps, magnesium, and microelements (potassium iodine, selenium and/or chromium);
F5, which comprises therapeutically effective amounts of one or more of: taurine, electrolytes, honokiol, B vitamins and piperine; and
F6, which comprises therapeutically effective amounts of one or more of: ashwagandha, L-theanine, saffron, Gingko biloba extract, and piperine.

8. The method of claim 7, comprising administering F1, F1-F2, F1-F3, F1-F4, F1-F5 or F1-F6.

9. The method of claim 1, further comprising, prior to treating the subject, diagnosing the subject with ASD.

10. The method of claim 1, further comprising prescribing a low-sugar or low-carbohydrate diet to the subject.

11. The method of claim 1, further comprising assessing the effect of the treatment on the reduction in severity of ASD in the subject, and adjusting the treatment if the assessment indicates said adjustment.

12. The method of claim 11, wherein the assessment indicates that the treatment with the antibiotic, antiviral or antifungal has reduced symptoms caused by a bacterial, viral or fungal infectious agent such that the subject no longer requires the antibiotic, antiviral or antifungal, and ceasing treatment with the antibiotic, antiviral or antifungal while continuing treatment with the one or more adjuvant supplement compositions.

13. A supplement composition selected from:
a supplement composition comprising therapeutically-effective amounts of one or more ingredients selected from curcumin, N-acetylcysteine, resveratrol, zinc and piperine;
a supplement composition comprising therapeutically effective amounts of one or more of: monolaurin, L-lysine, caprylic acid, pentadecanoic acid, and vitamin D;
a supplement composition comprising therapeutically effective amounts of one or more of: Lactobacilli, Bifidobacteria, Bacilli, and banana powder;
a supplement composition comprising therapeutically effective amounts of one or more of: berberine, diindolymethane (DIM), Cordyceps, magnesium, and microelements (potassium iodine, selenium and/or chromium);
a supplement composition comprising therapeutically effective amounts of one or more of: taurine, electrolytes, honokiol, B vitamins and piperine; and
a supplement composition comprising therapeutically effective amounts of one or more of: ashwagandha, L-theanine, saffron, Gingko biloba extract, and piperine.

14-18. (canceled)
